(19) United States
(12) Patent Application Publication
(10) Pub. No.: US 2017/0119727 A1
(43) Pub. Date: May 4, 2017

(54) A THERAPEUTIC PROTOCOL FOR TREATING OVARIAN CANCER

(71) Applicant: Bionomics Limited, Thebarton (AU)

(72) Inventors: Gabriel Kremmidiotis, Flagstaff Hill (AU); David C. BIBBY, Williams Landing (AU); Danny RISCHIN, East Melbourne (AU)

(73) Assignee: Bionomics Limited, Thebarton (AU)

(21) Appl. No.: 15/315,025

(22) PCT Filed: May 30, 2014
(86) PCT No.: PCT/AU2014/050055

§ 371 Date: Nov. 30, 2016

(51) Int. Cl. A61K 31/343; A61K 31/555; A61K 31/7068

(52) U.S. Cl. CPC A61K 31/343; A61K 31/7068; A61K 31/555

(57) ABSTRACT

The present invention is directed to a pharmaceutical combination for treating proliferative disease comprising a compound of formula (I), or a salt, solvate or prodrug thereof, carboplatin or cisplatin, and gemcitabine. The present invention is further directed to a therapeutic protocol to manage ovarian cancer treatment in a female subject involving the administration of the combination to an ovarian cancer subject with recurrent or persistent ovarian cancer following treatment with platinum-based therapy. Kits comprising the combination as well as the use of the combination in the treatment of a proliferative disease are further contemplated.

### BACKGROUND

Field

The present specification relates to a therapeutic protocol for the treatment of proliferative diseases including ovarian cancer.

Description of Prior Art

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Cancer is typically treated with either chemotherapy and/or radiation therapy. Whilst often effective to destroy a significant amount of tumor cells, such therapies often leave behind a number of tumor cells that is resistant to the treatment. These resistant cells can proliferate to form new tumors that are then resistant to treatment. As a result, the constant use of known combinations of chemotherapeutic drugs has given rise to multidrug resistant (‘MDR’) tumor cells.

The mode of proliferative diseases, such as tumors, is multi-factorial. For instance, research over the last forty years has led to the realization that cytotoxic agents (or anti-proliferative agents) includes anti-metabolic agents which interfere with microtubule formulation, alkylating agents which are able to cross-link DNA, platinum based agents which are able to interfere with DNA alkylation by blocking DNA replication, antitumor antibiotic agents, topoisomerase inhibitors, etc. In the treatment of such diseases drugs with different mechanisms may be combined (i.e., combination therapies) with beneficial effects including the effective treatment of MDR tumor cells and to minimize side effects such as undesirable cytotoxicity. The difficulty here is that not all known antiproliferative agents provide useful or beneficial effects in combination and accordingly research in many laboratories is presently focused on developing new and useful anti-proliferative combination partners.

Ovarian cancer is one of the most sensitive solid tumors to antineoplastic chemotherapy. Responses are typically expected in over 80% of patients who undergo surgical resection of the tumor mass in combination with standard platinum-based chemotherapy. However, despite this statistic, the majority of patients with advanced ovarian cancer will ultimately relapse and develop drug-resistant disease for which there is no curative treatment. There is, therefore, a need for effective treatment options for relapsed disease.

### SUMMARY

The present specification teaches a therapeutic protocol comprising the use of a combination of compounds wherein one compound is selected from a class of substituted benzofuran tubulin polymerisation inhibitors and two other compounds are anti-proliferative agents for treating proliferative diseases such as ovarian cancer, including recurrent or persistent ovarian cancer.

Enabled herein is a pharmaceutical combination for treating a proliferative disease comprising: (a) a compound of formula (I) or a salt, solvate or prodrug thereof;

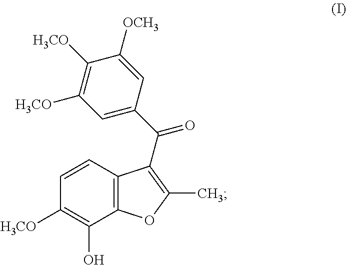 [SMILES: COc1cc(C(=O)c2c(C)oc3c(O)c(OC)ccc23)cc(OC)c1OC]

(b) carboplatin or cisplatin; and
(c) gemcitabine.

The present specification is instructional for a method for treating a proliferative disease including the step of administering to a patient in need thereof: (a) a compound of formula (I) or a pharmaceutically acceptable salt, solvate or prodrug thereof;

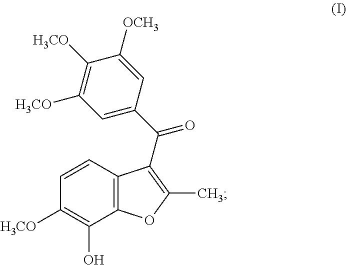 [SMILES: COc1cc(C(=O)c2c(C)oc3c(O)c(OC)ccc23)cc(OC)c1OC]

(b) carboplatin or cisplatin; and
(c) gemcitabine.

Taught herein is the use of: (a) a compound of formula (I) or a pharmaceutically acceptable salt, solvate or prodrug thereof:

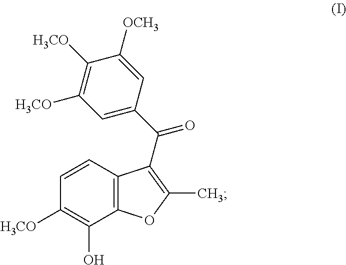 [SMILES: COc1cc(C(=O)c2c(C)oc3c(O)c(OC)ccc23)cc(OC)c1OC]

(b) carboplatin or cisplatin; and
(c) gemcitabine,
in the manufacture of a medicament for the treatment of a proliferative disease.

Further taught herein is: (a) a compound of formula (I) or a pharmaceutically acceptable salt, solvate or prodrug thereof:

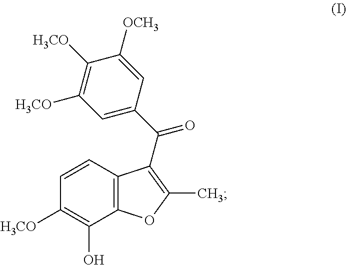 [SMILES: COc1cc(C(=O)c2c(C)oc3c(O)c(OC)ccc23)cc(OC)c1OC]

(b) carboplatin or cisplatin; and
(c) gemcitabine,
for use in the treatment of a proliferative disease.

Enabled herein is a pharmaceutical composition comprising (a) a compound of formula (I) or a pharmaceutically acceptable salt, solvate or prodrug thereof:

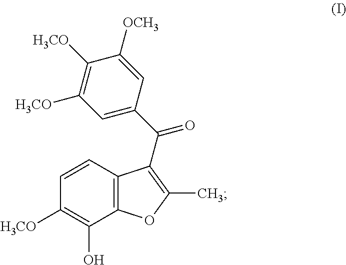 [SMILES: COc1cc(C(=O)c2c(C)oc3c(O)c(OC)ccc23)cc(OC)c1OC]

(b) carboplatin or cisplatin; and
(c) gemcitabine.

The pharmaceutical composition may further comprise one or more pharmaceutically acceptable carriers, diluents and/or excipients.

The present specification teaches a kit for the treatment of a proliferative disease comprising:
(a) a compound of formula (I) or a pharmaceutically acceptable salt, solvate or prodrug thereof:

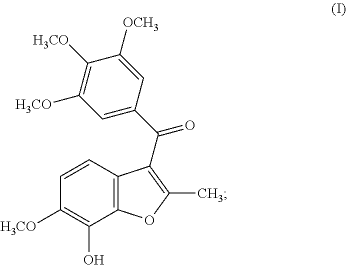 [SMILES: COc1cc(C(=O)c2c(C)oc3c(O)c(OC)ccc23)cc(OC)c1OC]

(b) carboplatin or cisplatin;
(c) gemcitabine; and
(d) instructions for use of (a), (b) and (c) in combination.

In an embodiment, the present specification enables a therapeutic protocol to manage ovarian cancer treatment in a female subject, said protocol comprising:
(a) selecting an ovarian cancer subject which has had recurrent or persistent ovarian cancer following treatment with platinum-based therapy;
(b) administering a combination of effective amounts of (a) a compound of formula (I); (b) carboplatin or cisplatin; and (c) gemcitabine for a defined period of time; and then
(c) administering effective amounts of a compound of formula (I) for a defined period of time.

Surprisingly, it has been found that the effects in treating proliferative diseases with a combination which comprises: (a) a compound of formula (I) or a pharmaceutically acceptable salt, solvate or prodrug thereof:

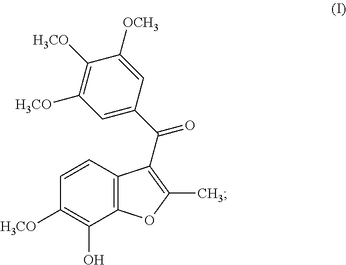 [SMILES: COc1cc(C(=O)c2c(C)oc3c(O)c(OC)ccc23)cc(OC)c1OC]

(b) carboplatin or cisplatin; and
(c) gemcitabine,
is greater than the effects that can be achieved with (b) and (c) together without (a). That is, in an embodiment, the present triple combination may possess a supra additive or synergistic effect, or provide a beneficial additive effect in anti-cancer therapy.

### BRIEF DESCRIPTION OF THE FIGURES

FIG. 1—depicts a graph of % perfusion control against an amount of compound (mg/kg) in relation to comparative levels of vascular shutdown (reduction in tumor perfusion) between CA4P and compound Example 2 of the present invention.

FIG. 2—depicts a graph of Tumor Volume ratio (Day*/Day 1) against time (Days) in relation to tumor growth inhibition of compound Example 2 in Balb/c nu/nu mice bearing MDA-MB-231 orthotopic breast solid tumors.

FIG. 3—depicts a graph of tumor volume against time (Days) in relation to tumor growth inhibition of compound Example 2 in combination with carboplatin and gemcitabine in Balb/c nu/nu mice bearing cisplatin resistant A2780Cis orthotopic ovarian solid tumors.

FIG. 4—depicts a schematic of the dosing and biomarker sampling timepoints of patients recruited for the phase I clinical trial of compound Example 2 in combination with carboplatin and gemcitabine.

FIG. 5—depicts four graphs of plasma concentrations of ferritin, interleukin-8, interleukin-16 and macrophage inflammatory protein-1β against the biomarker sampling timepoint. Absolute plasma biomarker concentration represented relative to pre-treatment baseline concentration. The statistical significance between pre- and post-treatment biomarker concentration was determined by paired, two-tailed t-test where P values <0.05 were considered significant.

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used in the subject specification, the singular forms “a”, “an” and “the” include plural aspects unless the context clearly dictates otherwise. Thus, for example, reference to a “formulation” includes a single formulation, as well as two or more formulations; reference to “an agent” includes a single agent, as two or more agents; reference to “the disclosure” includes a single and multiple aspects taught by the disclosure; and so forth. Aspects taught and enabled herein are encompassed by the term “invention”. All aspects are enabled within the width of the present invention.

The present specification teaches a therapeutic protocol to manage ovarian cancer treatment in a female subject, said protocol comprising:
(a) selecting an ovarian cancer subject which has had recurrent or persistent ovarian cancer following treatment with platinum-based therapy;
(b) administering a combination of effective amounts of (a) a compound of formula (I); (b) carboplatin or cisplatin; and, (c) gemcitabine for a defined period of time; and then
(c) administering effective amounts of a compound of formula (I) for a defined period of time.

Enabled herein is a pharmaceutical combination for treating a proliferative disease comprising: (a) a compound of formula (I) or a salt, solvate or prodrug thereof; (b) carboplatin or cisplatin; and (c) gemcitabine.

### Combination Partner (a); Compound of Formula (I)

A compound of formula (I) (2-Methyl-7-hydroxy-3-(3,4,5-trimethoxybenzoyl)-6-methoxybenzofuran) can be prepared by the synthetic methodology described in PCT/AU2007/000101 (WO 07/087684), the entire contents of which are included herein by reference.

A compound of formula (I) is observed to be potent tubulin polymerization inhibitor (TPI). TPI compounds are important in the treatment of cancers primarily as a result of their capacity to selectively shut down blood flow through a tumor. Compounds that inhibit tumor blood flow are generally referred to as vascular disrupting agents (VDAs) (Tozer, G. M.; Kanthou, C.; Baguley, B. C. Nature Rev., Vol. 5, 2005, 423). TPIs are VDAs because they inhibit a certain cell signalling pathway associated with microtubules, leading to interference in the regulation of the cytoskeleton of the endothelial cells that line the blood vessels of the tumor. As a result, these usually flat cells become more rounded, ultimately occluding blood flow through the vessels. The selectivity associated with these agents results from the fact that tumor vasculature is weaker and more prone to collapse than normal vasculature. Nonetheless, a number of the dose limiting toxicities associated with VDAs is due to a reduction in blood flow in healthy tissues. An important aspect of a compound of formula (I) is the combination of the specific C-6 and C-7 substituents together with the C-2 methyl group which appears to confer greater potency and selectivity when compared to other structurally related TPI compounds. In this compound, selectivity is not merely reliant on the predisposition of tumour vasculature towards collapse when challenged with the VDA but on a capacity of the VDA to distinguish between tumor endothelial cells and normal endothelial cells. Normal endothelial cells, found in healthy tissues, are in a “quiescent” state and tumor endothelial cells are in an “activated” state. Most VDAs do not distinguish between these two states, for example, Combretastatin A4 is equally potent against quiescent and activated endothelial cells. However, a compound of formula (I) shows high potency towards tumor endothelial cells (activated) over normal endothelial cells (quiescent).

It will be appreciated that a compound of formula (I) can be administered to a subject as a pharmaceutically acceptable salt thereof. Suitable pharmaceutically acceptable salts include, but are not limited to salts of pharmaceutically acceptable inorganic acids such as hydrochloric, sulfuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulfanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts e.g. sodium or potassium salts, or alkyl esters (e.g. methyl, ethyl) of the phosphate group.

It will also be appreciated that any compound that is a prodrug of a compound of formula (I) is also within the scope of this specification. The term “pro-drug” is used in its broadest sense and encompasses those derivatives that are converted in vivo to a compound of formula (I). Such derivatives would readily occur to those skilled in the art, and include, for example, compounds where the free hydroxy group at the C-7 position is converted into an ester, such as an acetate or phosphate ester. Procedures for esterifying, e.g. acylating, a compound of formula (I) are well known in the art and may include treatment of the compound with an appropriate carboxylic acid, anhydride or chloride in the presence of a suitable catalyst or base. A particularly preferred prodrug is a disodium phosphate ester. The disodium phosphate ester of the compound of the invention may be useful in increasing the solubility of the compound. This, for instance, may allow for delivery of the compound in a benign vehicle like saline. The disodium phosphate ester may be prepared in accordance with the methodology described in Pettit, G. R., et al, Anticancer Drug Des., 1995, 10, 299. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers).

Accordingly, in an embodiment, a compound of formula (I) is a compound represented by the following:

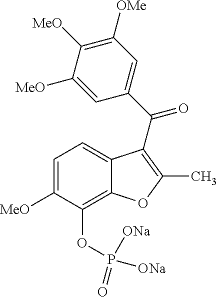

A compound of formula (I) (or a prodrug thereof) may be in crystalline form either as the free compound or as a solvate (e.g. hydrate) and it is intended that both forms are within the scope of the present invention. Methods of solvation are generally known within the art.

Combination Partner (b)—Carboplatin or Cisplatin

In an embodiment, the combination partner (b) is carboplatin also known as cis-Diammine (1,1-cyclobutane dicarboxylato) platinum (II) (Paraplatin or Paraplatin-AQ).

In an embodiment the combination partner (b) is cisplatin also known as cis-platinum (II)-diaminedichloride (Platinol).

Combination Partner (c)—Gemcitabine

In an embodiment, the combination partner (c) is gemcitabine (Gemzar).

These above anti-proliferative combination partners (b) and (c) are known in the art and their synthesis would also be known to those skilled in the art.

As used herein the term “ovarian cancer” broadly encompasses any neoplastic disease including those which are potentially malignant (pre-cancerous) or malignant (cancerous) of the ovary.

In an embodiment, the combination may be used in the treatment of ovarian tumors. More than 90% of ovarian cancers are classified as epithelial cancers and the majority of these tumors are thought to arise in the fallopian tube. Some evidence suggests that the endothelium, gastro-intestinal tract and the ovarian surface epithelium itself could also be the source of some ovarian cancers. The present specification teaches the treatment of ovarian cancers arising from both or either one of the above postulated sources.

Despite the differences in ovarian cancers, the standard treatment regimen is the same for all ovarian cancer subjects. Primary treatment for ovarian cancer involves surgical removal of the tumor mass followed by platinum-based chemotherapy. Although the majority of ovarian cancer subjects initially respond well to primary treatment, most subjects will eventually relapse. There are no curative treatments currently available for recurrent disease.

In the present context the term “platinum-based chemotherapy” is used synonymously with the term “platinum-based therapy” and broadly encompasses any therapy that includes cisplatin or carboplatin.

In an embodiment, the combination may be used in a therapeutic protocol for the treatment of recurrent or persistent ovarian cancer.

As used herein the term “recurrent or persistent” broadly encompasses any relapse in the disease of an ovarian cancer subject that occurs following treatment with a platinum-based therapy. This is inclusive of ovarian cancer subjects that have platinum-sensitive recurrence, platinum-refractory recurrence or platinum-resistant recurrence. “Platinum-sensitive recurrence” is intended to mean a disease recurrence that occurs more than 6 months after the last dose of a platinum-based therapy. “Platinum-refractory recurrence” is intended to mean a disease recurrence that occurs within 6 months after the last dose of a platinum-based therapy. “Platinum-resistant recurrence” is intended to mean a disease recurrence that occurs before the completion of a course of platinum-based therapy.

Enabled herein is a therapeutic protocol to manage ovarian cancer treatment in a female subject, the protocol comprising:
(i) selecting an ovarian cancer subject which has had platinum-based therapy and has had recurrent or persistent disease.
(ii) administering a combination of effective amounts of (a) a compound of formula (I) or a pharmaceutically acceptable salt, solvate or prodrug thereof with (b) carboplatin or cisplatin and (c) gemcitabine for a defined period of time; and then
(iii) administering effective amounts of a compound of formula (I) for a defined period of time.

In an embodiment, ovarian cancer subjects are selected following the first relapse following the cessation of primary treatment.

In an embodiment, ovarian cancer subjects are selected following the second relapse following the cessation of secondary treatment.

In an embodiment, ovarian cancer subjects are selected following any additional relapse where it is deemed that the combination therapy will improve patient outcome.

The present specification also teaches a method of treating ovarian cancer comprising the administration of an effective amount of (a) a compound of formula (I) or a pharmaceutically acceptable salt, solvate or prodrug thereof with combination partners (b) and (c) as a triple combination for a defined period of time and then administering an effective amount of a compound of formula (I) or a pharmaceutically acceptable salt, solvate or prodrug thereof.

An “effective amount” is intended to mean that the amount of each combination partner, when administered to a mammal (in particular a human) in need of such treatment, is sufficient to effect treatment for ovarian cancer. Thus, for example, a therapeutically effective amount of a compound of the formula (I) (or a pharmaceutically acceptable salt, solvate, or prodrug thereof) may be a quantity sufficient to synergise or potentiate the activity of the at least one of (b) or (c) or both such that the cancer state is reduced or alleviated.

In an embodiment, the effective amount of a compound of formula (I) is from 10 to 20 mg/m². Reference to “from 10 to 20 mg/m²” means 10, 11, 12, 13, 14, 15, 16, 17, 18, 19 or 20 mg/m².

In an embodiment, the effective amount of combination partner (b) is AUC4.

In an embodiment, the effective amount of combination partner (c) is 500 to 1500 mg/m². Reference to “500 to 1500 mg/m²” means 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499 or 1500 mg/m².

The terms “treatment” or “treating” as used herein are intended to include at least partially attaining the desired effect, or delaying the onset of, or inhibiting the progression of, or halting or reversing altogether the onset or progression of the particular cancer being treated.

Without wanting to be bound by any particular theory or mode of action, it is proposed that the tumor vascular disruption effect caused by a compound of formula (I) is transient (at least in some tumors) with tumour re-vascularisation occurring after around 48 hrs. It is considered that the synergistic or additive effect proposed by the present combination is the inhibition or delay of this re-vascularisation event and tumor recovery from the initial disruption with a compound of formula (I).

In an embodiment the defined period of administration of a compound of formula (I) with combination partners (b) and (c) is from 1 to 6 cycles of drug therapy. Reference to “from 1 to 6 cycles” means 1, 2, 3, 4, 5 or 6 cycles.

The administration of the pharmaceutical combination of the present invention may result not only in a beneficial effect, e.g., a synergistic therapeutic effect, for instance, with regard to alleviating, delaying progression of or inhibiting the symptoms, but also in further beneficial effects. Such other effects may include fewer side effects, an improved quality of life or a decreased morbidity, compared with a monotherapy applying only one of the pharmaceutically active ingredients used in the combination of the present invention.

A further benefit of the subject protocol is that lower doses of the active ingredients of the combination can be used. The dosages need not only be smaller but may also be applied less frequently, which may diminish the incidence or severity of side effects.

In an embodiment, a therapeutically effective amount of each of the combination partners of the combination of the invention is administered simultaneously or sequentially and in any order, and the combination partners are administered separately or as a fixed combination.

For example, the method of preventing or treating proliferative diseases according to the invention may comprise: (i) administration of the first agent (a) in free or pharmaceutically acceptable salt form; followed by (ii) administration of an agent (b) and then (c) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single (e.g., fixed) combination forms. Furthermore, the term administering also encompasses the use of a pro-drug of a combination partner that convert in vivo to the combination partner as such. The instant specification embraces all such regimens of simultaneous or alternating treatment and the term “administering” is to be interpreted accordingly.

In an embodiment, each cycle of drug therapy is 21 days with a compound of formula (I) administered at days 2 and 9, combination partner (b) at day 1 and combination partner (c) at days 1 and 8.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A physician of ordinary skill can readily determine and prescribe the effective amount of the single active ingredients required to treat the disease state.

Daily dosages for combination partners (a), (b) and (c) will, of course, vary depending on a variety of factors, e.g., the compound chosen, the particular disease to be treated and the desired effect. In general, however, satisfactory results may be achieved on administration of a compound of formula (I) at daily dosage rates of about 0.03 to 5 mg/kg per day as a single dose or in divided doses. Reference to “0.03 to 5 mg/kg” means 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.99, 3.00, 3.01, 3.02, 3.03, 3.04, 3.05, 3.06, 3.07, 3.08, 3.09, 3.10, 3.11, 3.12, 3.13, 3.14, 3.15, 3.16, 3.17, 3.18, 3.19, 3.20, 3.21, 3.22, 3.23, 3.24, 3.25, 3.26, 3.27, 3.28, 3.29, 3.30, 3.31, 3.32, 3.33, 3.34, 3.35, 3.36, 3.37, 3.38, 3.39, 3.40, 3.41, 3.42, 3.43, 3.44, 3.45, 3.46, 3.47, 3.48, 3.49, 3.50, 3.51, 3.52, 3.53, 3.54, 3.55, 3.56, 3.57, 3.58, 3.59, 3.60, 3.61, 3.62, 3.63, 3.64, 3.65, 3.66, 3.67, 3.68, 3.69, 3.70, 3.71, 3.72, 3.73, 3.74, 3.75, 3.76, 3.77, 3.78, 3.79, 3.80, 3.81, 3.82, 3.83, 3.84, 3.85, 3.86, 3.87, 3.88, 3.89, 3.90, 3.91, 3.92, 3.93, 3.94, 3.95, 3.96, 3.97, 3.98, 3.99, 4.00, 4.01, 4.02, 4.03, 4.04, 4.05, 4.06, 4.07, 4.08, 4.09, 4.10, 4.11, 4.12, 4.13, 4.14, 4.15, 4.16, 4.17, 4.18, 4.19, 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39, 4.40, 4.41, 4.42, 4.43, 4.44, 4.45, 4.46, 4.47, 4.48, 4.49, 4.50, 4.51, 4.52, 4.53, 4.54, 4.55, 4.56, 4.57, 4.58, 4.59, 4.60, 4.61, 4.62, 4.63, 4.64, 4.65, 4.66, 4.67, 4.68, 4.69, 4.70, 4.71, 4.72, 4.73, 4.74, 4.75, 4.76, 4.77, 4.78, 4.79, 4.80, 4.81, 4.82, 4.83, 4.84, 4.85, 4.86, 4.87, 4.88, 4.89, 4.90, 4.91, 4.92, 4.93, 4.94, 4.95, 4.96, 4.97, 4.98, 4.99 or 5 mg/kg.

A compound of formula (I) and combination partners (b) and (c) may be administered by any conventional route, in particular enterally, e.g., orally, e.g., in the form of tablets, capsules, drink solutions or parenterally, e.g., in the form of injectable solutions or suspensions. Suitable unit dosage forms for oral administration comprise from about 0.02 to 50 mg active ingredient, e.g. combination partner (a), (b), and/or (c) together with one or more pharmaceutically acceptable diluents or carriers therefore. Reference to “0.02 to 50 mg” means 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg.

A compound of formula (I) may be administered to a human in a daily dosage range of 0.5 to 1000 mg. Reference to “0.5 to 1000 mg” means 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999 or 1000 mg. Suitable unit dosage forms for oral administration comprise from about 0.1 to 500 mg active ingredient, together with one or more pharmaceutically acceptable diluents or carriers therefore. Reference to “0.1 to 500 mg” means 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 mg.

The pharmaceutical compositions for separate administration of a compound of formula (I) with partners (b) and (c) or for the administration in a fixed combination (i.e., a composition comprising both (a) and (b)) according to the invention, may be prepared in a manner known in the art and are those suitable for enteral, such as oral or rectal, and parenteral administration to mammals (warm-blooded animals), particularly humans, comprising a therapeutically effective amount of at least one pharmacologically active combination partner alone, e.g., as indicated above, or in combination with one or more pharmaceutically acceptable carriers or diluents, especially suitable for enteral or parenteral application.

Suitable pharmaceutical compositions contain, e.g., from about 0.1% to about 99.9%, preferably from about 1% to about 60%, of the active ingredient(s). Reference to “about 1% to about 60%” means 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60%.

The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the subject. Compositions include those suitable for oral, rectal, nasal, topical (including buccal and sublingual), vaginal or parental (including subcutaneous, intramuscular, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

Compositions for rectal administration may be presented as a suppository with a suitable base comprising, for example, cocoa butter, glycerin, gelatine or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring.

Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The present specification teaches a kit of parts comprising:
(i) a compound of formula (I) or a pharmaceutically acceptable salt, solvate or prodrug thereof in a first unit dosage form; and
(ii) carboplatin or cisplatin;
(iii) gemcitabine; and
(iv) instructions for use of (i), (ii) and (iii) in combination for treating a proliferative disease.

In an embodiment, the therapeutic kit for the treatment of ovarian cancer in a female subject at first, second or subsequent relapse after platinum-based chemotherapy comprising (i), (ii), (iii) and (iv) components, the kit in a form to dispense component (i) in a dosage amount from 10 to 20 mg/m². Reference to “10 to 20 mg/m²” means 10, 11, 12, 13, 14, 15, 16, 17, 18, 19 or 20 mg/m². Component (ii) in a dosage amount of AUC4, and component (iii) is an dosage amount of from 500 to 1500 mg/m². Reference to “500 to 1500 mg/m² means 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499 or 1500 mg/m².

In an embodiment, instruction for use of (i), (ii) and (iii) components for use in treatment of ovarian cancer in a female subject at first, second or subsequent relapse after platinum-based chemotherapy, wherein (i), (ii) and (iii) components are administered to the subject for a defined period followed by component (i) for a defined period.

Further taught herein is a commercial package comprising the combination according to the present invention together with instructions for simultaneous, separate or sequential use.

In an embodiment, the (commercial) product is a commercial package comprising as active ingredients the combination according to the present specification (in the form of two or three or more separate units of the components as described above), together with instructions for simultaneous, separate or sequential use, of any combination thereof, in the treatment of the disease states as mentioned herein.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Synthetic Protocols

### Preparation of 2-Bromo-7-acetoxy-3-(3,4,5-trimethoxybenzoyl)-6-methoxybenzofuran

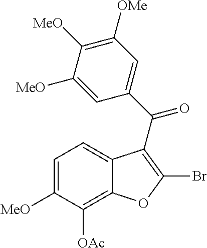 [SMILES: COc1cc(C(=O)c2c(Br)oc3c(OC(C)=O)c(OC)ccc23)cc(OC)c1OC]

### Step 1: 2-t-Butyldimethylsilyl-3-(t-butyldimethylsilyloxymethylene)-6-methoxy-7-isopropoxybenzofuran (Larock coupling)

A suspension of 2-isopropoxy-3-methoxy-5-iodophenol (4.41 mmol), 1-(tert-butyldimethylsilyl)-3-(tert-butyldimethylsilyloxy)propyne (1.5 g, 5.28 mmol), lithium chloride (189 mg, 4.45 mmol) and sodium carbonate (2.34 g, 22.08 mmol) in dry dimethylformamide (5 mL) at 100° C. was deoxygenated 4 times by evacuation and backfilling with nitrogen. Palladium acetate (135 mg, 0.60 mmol) was added and the reaction vessel was degassed twice with nitrogen. The reaction mixture was then stirred at this temperature for 4 hours (tlc) and the solvent was removed by distillation under vacuum. The residue was dissolved in ethyl acetate (75 mL), stirred well, filtered and treated with triethylamine (5 mL). The solution was concentrated onto silica gel (10 g) and purified by flash chromatography (silica gel, eluent=hexane/diethyl ether/triethylamine; 95:5:1%) to afforded the title compound as a yellow oil (1.45 g, 96%); ¹H NMR (300 MHz, CDCl₃) δ 7.24 (d, 1H, J=8.45 Hz), 6.88 (d, 1H, J=8.47 Hz), 4.80 (s, 2H, CH₂), 4.73 (m, 1H), 3.88 (s, 3H, OMe), 1.36 (d, 6H, J=6.17 Hz), 0.94 (s, 9H), 0.92 (s, 9H), 0.35 (s, 6H), 0.12 (s, 6H).

### Step 2: 2-t-Butyldimethylsilyl-3-formyl-6-methoxy-7-isopropoxybenzofuran

To a solution of 2-t-butyldimethylsilyl-3-(t-butyldimethylsilyloxymethylene)-6-methoxy-7-isopropoxybenzofuran (2.69 mmol) in methanol (100 mL) was added concentrated hydrochloric acid (200 μL) and the reaction was stirred for 30 minutes (monitored by tlc), quenched with triethylamine (2 mL) and the solvent removed by distillation under vacuum. The residue was dissolved in dichloromethane (20 mL), washed with water (10 mL), dried over magnesium sulfate, concentrated under vacuum and co-distilled with toluene (20 mL). The crude product was dissolved in dry dichloromethane (4 mL) and added to a stirred solution of Collin's reagent (chromium trioxide (1.01 g), pyridine (1.65 mL) in dry dichloromethane (30 mL)). The suspension was stirred for 10 minutes, filtered and the residue washed with diethyl ether (20 mL). The filtrate was concentrated onto silica (10 g) and purified by flash chromatography (silica gel, eluent=hexane/diethyl-ether/triethylamine (90:9:1) to afford the title compound as a light yellow oil (503 mg, 48%); ¹H NMR (300 MHz, CDCl₃) δ 10.25 (s, 1H, CHO), 7.79 (d, 1H, J=8.45 Hz), 6.98 (d, 1H, J=8.46 Hz), 4.65 (m, 1H), 3.89 (s, 3H, OMe), 1.35 (d, 6H, J=6.17 Hz), 0.97 (s, 9H), 0.45 (s, 6H).

### Step 3: 2-t-Butyldimethylsilyl-3-(3,4,5-trimethoxybenzoyl)-6-methoxy-7-isopropoxybenzofuran

To a stirred solution of 3,4,5-trimethoxyiodobenzene (377 mg, 1.27 mmol) in dry tetrahydrofuran (1 mL) at −78° C. under nitrogen was added n-butyllithium (795 μL, 1.59 mmol, 2M solution in cyclohexane) and the reaction mixture was stirred at this temperature for 40 minutes. After this time a solution of 2-t-butyldimethylsilyl-3-formyl-6-methoxy-7-isoproxybenzofuran (1.07 mmol) in dry tetrahydrofuran (1 mL) was added to the reaction drop wise via syringe pipette. The reaction mixture was stirred at −60° C. for 20 minutes and then allowed to warm to 0° C., stirred for 10 minutes, quenched with saturated ammonium chloride solution (2 mL) and diluted with ethyl acetate (20 mL). The organic layer was washed with water (10 mL), dried over magnesium sulfate and the solvent was removed under vacuum to give a residue that was co-distilled with toluene. The crude product (908 mg) was dissolved in dry tetrahydrofuran (10 mL) and treated with 2,3-dichloro-5,6-dicyano-1,4-benzoquinone (900 mg, 1.59 mmol) was added. The reaction mixture was stirred at room temperature for 16 hours (monitored by tlc) and then loaded onto silica (10 g) and purified by flash chromatography (silica gel, eluent=hexane/diethyl ether/triethylamine, 90:9:1) to afford the title compound as a light yellow oil (498 mg, 69%); ¹H NMR (300 MHz, CDCl₃) δ 7.14 (s, 2H, benzoyl Hs), 6.81 (d, 1H, J=8.64 Hz), 6.77 (d, 1H, J=8.64 Hz) 4.74 (m, 1H), 3.93 (s, 3H, OMe), 3.86 (s, 3H, OMe), 3.78 (s, 6H, 2×OMe), 1.39 (d, 6H, J=6.14 Hz), 1.01 (s, 9H), 0.26 (s, 6H).

### Step 4: 2-(tert-butyldimethylsilyloxy)-7-acetoxy-3-(3,4,5-trimethoxybenzoyl)-6-methoxybenzofuran

To a stirred solution of 2-(t-butyldimethylsilyloxy)-7-isopropoxy-3-(3,4,5-trimethoxybenzoyl)-6-methoxy-benzofuran (160 mg, 0.31 mmol) in dry DCM (2 mL) at room temperature under nitrogen was added solid aluminium trichloride (83 mg, 0.62 mmol) and the reaction mixture was stirred for 15 minutes (monitored by tlc). The reaction was quenched with a saturated solution of ammonium chloride, extracted with dichloromethane and dried over magnesium sulfate. The solvent was removed by distillation and residue was dried by azeotropic removal of water with toluene. The crude product was dissolved in pyridine (2 mL), acetic anhydride (1 mL) was added and reaction mixture was stirred for 2 hours at room temperature. The solvent was distilled under vacuum and the residue was loaded onto silica gel (1 g) and purified by column chromatography (silica gel, eluent, hexane:diethyl-ether; 80:20) (134 mg, 84%); ¹H NMR (300 MHz, CDCl₃) δ 7.14 (s, 2H, benzoyl Hs), 6.98 (d, 1H, J=8.72 Hz), 6.85 (d, 1H, J=8.72 Hz), 3.93 (s, 3H, OMe), 3.86 (s, 3H, OMe), 3.80 (s, 6H, 2×OMe), 2.41 (s, 3H), 0.99 (s, 9H), 0.25 (s, 6H).

### Step 5: 2-Bromo-7-acetoxy-3-(3,4,5-trimethoxybenzoyl)-6-methoxybenzofuran

To a stirred solution of 2-t-butyldimethylsilyl-7-acetoxy-3-(3,4,5-trimethoxybenzoyl)-6-methoxybenzofuran (120 mg, 0.44 mmol) in 1,2-dichloroethane (1 mL) at room temperature under nitrogen was added bromine (12 μl, 0.44 mmol) drop wise and the reaction mixture was stirred at this temperature for 10 minutes. After this time the reaction was quenched with saturated sodium thiosulfate solution, extracted with ethyl acetate (20 mL), dried over magnesium sulfate and the solvent removed by distillation under vacuum. The crude product was purified by silica gel column chromatography (eluent=Hexane:diethyl ether; 8:2-7:3) to afford the title compound as a colourless crystalline solid (91 mg, 81%); ¹H NMR (300 MHz, CDCl₃) δ 7.40 (d, 1H, J=8.70 Hz), 7.14 (s, 2H, benzoyl-Hs), 6.98 (d, 1H, J=8.75 Hz), 3.94 (s, 3H, OMe), 3.89 (s, 3H, OMe), 3.86 (s, 6H, 2×OMe), 2.43 (s, 3H); ¹³C NMR (75 MHz, CDCl₃) δ 187.95 (CO), 167.71, 152.75, 149.54, 147.49, 142.59, 131.92, 131.80, 123.91, 121.84, 119.89, 117.72, 109.89, 106.92, 60.69, 56.61, 56.00, 20.09.

### Example 1

### Preparation of 2-Methyl-7-hydroxy-3-(3,4,5-trimethoxybenzoyl)-6-methoxybenzofuran

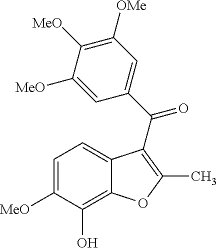 [SMILES: COc1cc(C(=O)c2c(C)oc3c(O)c(OC)ccc23)cc(OC)c1OC]

### Preparation A

To a stirred solution of 2-Bromo-7-acetoxy-3-(3,4,5-trimethoxybenzoyl)-6-methoxybenzofuran (20 mg, 0.042 mmol), methyl-boronic acid (40 mg, 0.67 mmol), in 1,4-dioxane (2 mL) at 90° C. was added tetrakis-triphenylphosphine palladium (11 mg, 0.01 mmol) followed by the addition of a solution of sodium bicarbonate (40 mg, 0.48 mmol) in distilled water (0.5 mL). The reaction mixture turned red after 5 minutes. After 2 hours (tlc) the reaction mixture was brought to room temperature and was added saturated ammonium chloride (2 mL) and diluted with dichloromethane (20 mL). The organic layer was separated and washed with water, dried over magnesium sulfate and the solvent was removed by distillation under vacuum. The residue was purified by PTLC (eluent=Dichloromethane/Methanol, 1:1) to give the title compound (acetate cleaved during reaction) as a fluffy white solid; (3 mg, 19%).

### Preparation B (Negishi Coupling)

To a stirred solution of zinc-bromide (592 mg, 2.63 mmol) in dry THF (1.5 mL) at 0° C. was added the solution of methyl lithium (1.6 M solution in diethyl-ether, 2.6 mL, 4.15 mmol) and the reaction mixture was stirred for 2 hours. Solid 2-bromo-7-acetoxy-3-(3,4,5-trimethoxybenzoyl)-6-methoxy-benzofuran (300 mg, 0.63 mmol) was added and the ether was removed under vacuum and to the rest suspension was added dichlorobis(triphenylphosphine)palladium catalyst (21 mg) and catalytic amount of copper (I) iodide. The reaction mixture was stirred at room temperature for 36 hours (monitored by tlc), quenched with saturated ammonium chloride solution and extracted with dichloromethane (10 mL), dried over magnesium sulfate and solvent distilled under vacuum and the product was purified by silica gel column (eluent=hexane/ethyl acetate; 8:2). The product was crystallized in methanol (106 mg, 46%); ¹H NMR (300 MHz, CDCl₃) δ 7.09 (s, 2H, benzoyl Hs), 6.93 (d, 1H, J=8.54 Hz), 6.83 (d, 1H, J=8.56 Hz), 5.70 (bs, 1H, OH), 3.93 (s, 3H, OMe), 3.92 (s, 3H, OMe), 3.83 (s, 6H, 2×OMe), 2.54 (s, 3H, 2-Me)

### Example 2

### Preparation of Disodium 6-methoxy-2-methyl-3-(3,4,5-trimethoxybenzoyl)benzofuran-7-yl phosphate

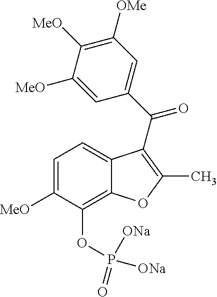

### Step 1: Dibenzyl 6-methoxy-2-methyl-3-(3,4,5-trimethoxybenzoyl)benzofuran-7-yl phosphate

To a mixture of 0.081 g (0.22 mmol) of (7-hydroxy-6-methoxy-2-methylbenzofuran-3-yl)(3,4,5-trimethoxyphenyl)methanone, 0.086 g (0.261 mmol) of carbon tetrabromide and 0.063 ml (0.283 mmol) of dibenzylphosphite in 2.5 ml of anhydrous acetonitrile 0.046 ml of anhydrous triethylamine was added drop wise at 0° C. under nitrogen atmosphere. The resulting mixture was stirred for 2 h at room temperature, then diluted to 20 ml with ethyl acetate, washed with water brine, dried over anhydrous magnesium sulfate, filtered off and evaporated to dryness under reduced pressure. The residue was purified by flash column chromatography (dichloromethane/ethyl acetate, 9:1) to give the title compound as a colorless foam (0.13 g, 94%); ¹H NMR (CDCl₃) δ 2.42 (s, 3H, Me-2); 3.83 (s, 1H, OMe); 3.93 (s, 3H, OMe); 5.33 (m, 4H, CH₂Ph); 6.89 (d, CH aromatic, J=8.7 Hz); 7.21 (dd, 1H, CH aromatic, J=8.72 Hz; J=1.2 Hz); 7.08 (s, 2H, CH aromatic); 7.29-7.43 (m, 10H, CH aromatic).

### Step 2: Disodium 6-methoxy-2-methyl-3-(3,4,5-trimethoxybenzoyl)benzofuran-7-yl phosphate

To a stirred solution of 0.122 g (0.193 mmol) of the product from Step 1 in 1 ml of anhydrous acetonitrile 0.075 ml (0.58 mmol) of bromotrimethylsilane was added at −5° C. under nitrogen atmosphere. The resulting mixture was stirred for 1 h at 0° C., then evaporated to dryness in vacuo. The residue was diluted to 5 ml with anhydrous methanol and pH of the solution was brought up about 10 by the addition of sodium methoxide. After evaporation of the resulting mixture under reduced pressure the solid residue was washed with anhydrous isopropanol (4×1.5 ml) and anhydrous ethanol (3×1.5 ml) and dried under vacuum to give 0.062 g (65% yield) of title compound as an colorless solid; ¹H NMR (D₂O) δ 2.37 (s, 3H, Me-2); 3.76 (s, 6H, OMe); 3.79 (s, 3H, OMe); 3.82 (s, 3H, OMe); 4.66 (s, H₂O); 6.93 (d, 1H, CH aromatic, J=8.6 Hz); 7.04 (d, 1H, CH aromatic, J=8.6 Hz); 7.10 (s, 2H, CH aromatic).

### Biological Data

### (A) (i) In Vitro Studies for Combination Partner (a)

TABLE 1
In Vitro Data for Compounds: These are the results for growth inhibition
studies of compounds using the Sulforhodamine B (SRB) or Systmex cell counting (CC)
assays. IC₅₀ is the concentration required to inhibit net cell growth by 50%. Entries 1-4
are provided for comparison, entry 5 is a compound of the invention (combination partner
(a)).

| | | | Cancer | |
|---|---|---|---|---|
| | | | cell | HUBECs^{c} |
| | | | line^{a}: | Tum: IC₅₀, nM |
| | Example/ | | IC₅₀, | Norm: IC₅₀, |
| Entry | Comparator | Structure | nM | nM |
| 1. | Comparator A | 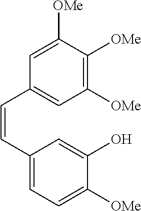 [SMILES: COc1ccc(/C=C\c2cc(OC)c(OC)c(OC)c2)cc1O] | 5 | Tum: 1-10 Norm: 1-10 |
| 2. | Comparator B | 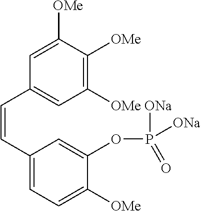 | 5 | Tum: 1-10 Norm: 1-10 |
| 3. | Comparator C Example 5 | 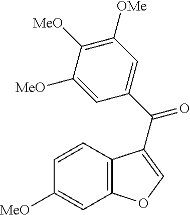 [SMILES: COc1ccc2c(C(=O)c3cc(OC)c(OC)c(OC)c3)coc2c1] | 55 | Turn: 10-100 Norm: 10-100 |
| 4. | Comparator D Example 3 | 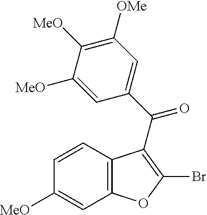 [SMILES: COc1ccc2c(C(=O)c3cc(OC)c(OC)c(OC)c3)c(Br)oc2c1] | 500 | Turn: 100-1000 Norm: 100-1000 |
| 5. | Example 1 | 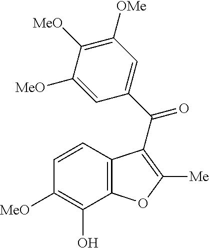 [SMILES: COc1cc(C(=O)c2c(C)oc3c(O)c(OC)ccc23)cc(OC)c1OC] | 2.0 | Turn: 0.1-1 Norm: 10-100 |

^{a}Unless otherwise stated the cancer cell line is MCF-7. For description of method of MCF-7 inhibition see: Verdier-Pinard, P et al. Mol. Pharmacol 1998, 53, 62-76
^{c}Human umbilical vein endothelial cells (HUVECs) tumour type activated endothelial cells (Turn) and normal quiescent type endothelial cells (Norm).

### General Description of Biological Experiments:

### Proliferation Assay—Quiescent Endothelium:

Human umbilical vein endothelial cells (CC-2519, Clonetics) were plated at 15000 cells/well in EBM2 (CC-3156, Clonetics)+0.5% FBS (CC-4101A, Clonetics)+GA-1000 (CC-4381A, Clonetics) in a 96 well plate in triplicate. Cells were cultured overnight at 37° C. 5% CO₂. Medium was subsequently replaced with fresh medium including the compound or negative control. Cells were cultured for a period of 48 hrs. An MTT assay was performed to measure changes in cell numbers. Briefly, 20 μl of MTT reagent was added to cells containing 100 μl of EBM2+0.5% FBS and incubated at 37° C. for 2 hours. Absorbance was measured at 492 nm.

### Proliferation Assay—Activated Endothelium:

Human umbilical vein endothelial cells (CC-2519, Clonetics) were plated at 2500 cells/well in EGM2 (CC-3162, Clonetics) in a 96 well plate in triplicate. Cells were cultured overnight at 37° C. 5% CO₂. Medium was subsequently replaced with fresh medium including the compound or negative control. Cells were cultured for a period of 48 hrs. An MTT assay was performed to measure changes in cell numbers. Briefly, 20 μl of MTT reagent was added to cells containing 100 μl of EGM2 and incubated at 37° C. for 2 hours. Absorbance was measured at 492 nm.

### (ii) In Vivo Studies for Combination Partner (a)

### Vascular Disruption Assay:

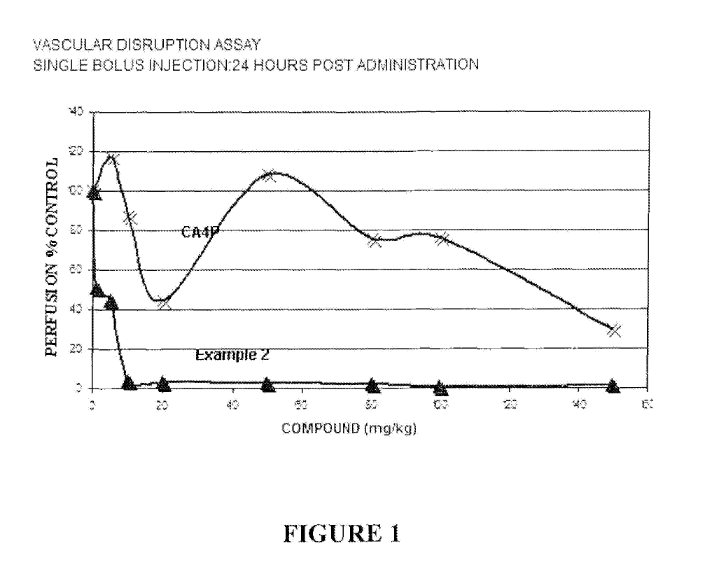

Female athymic BALB/c-nu/nu mice (nude mice) were used for this study. Mice were between 6-8 weeks old and were purchased from the Animal Resource Centre, Perth, Western Australia and allowed to acclimatize for a couple of days. All the animals were housed under pathogen-free conditions and cared for in accordance with Flinders University of South Australia and NH&MRC guidelines and the Australian Code of Practice for the care and use of animals for scientific purposes. The human breast cancer MDA MB 231 was grown as orthotopic xenografts in the mammary fat pad of nude mice. Each mouse was injected with 2×10⁶ cells in 50 μl Dulbecco's PBS subcutaneously just above the mammary fat pad, below the right forward limb. Tumours were selected for treatment when they reached a diameter of 100-150 mm³ (3 weeks after implantation). The test compound (Example 2) was dissolved in saline solution and injected intravenously at concentrations ranging from 150 mg/kg-1 mg/kg in a total volume of 400 ul. Tumour bearing animals were injected intravenously with 10 mg/kg Hoechst 33342, 24 hours after the injection of the test compound. Animals were euthanised 1 minute after the Hoechst 33342 injection. Tumours were recovered for histochemical analysis. Tumour perfusion analysis was performed by assessing the amount of Hoechst 33342 staining across an entire tumour cross-section. 10 micron sections of frozen tumour biopsies were viewed under an ultraviolet light filter. Using a 4× objective lens, 8-bit monochromatic images were captured in succession, representing the total area of the tumour section. Composite images of the total tumour section were generated by overlaying common areas of the monochromatic images. Hematoxylin and Eosin-Y staining of the same tumour section was performed to identify non-tumour regions. Non-tumour regions were mapped on Hoechst 33342 composite images and excluded from the quantitation analysis. Quantitation was performed by measuring the pixel area of Hoechst 33342 staining and the total pixel area of the tumour region. Perfusion was expressed as a percentage of Hoechst 33342 stained area to total tumour area (see FIG. 1).

### Tumour Growth Inhibition:

Balb/c nu/nu mice bearing MDA-MB-231 solid orthotopic tumours were treated with compound Example 2 at 40 mg/kg. Animals were i.v. dosed with a total of two cycles of Example 2 treatment. Each cycle was dosing on days and 8 followed by a three week no-dosing period. Tumour growth represented as a ratio to initial tumour volume is shown over a total of 72-days.

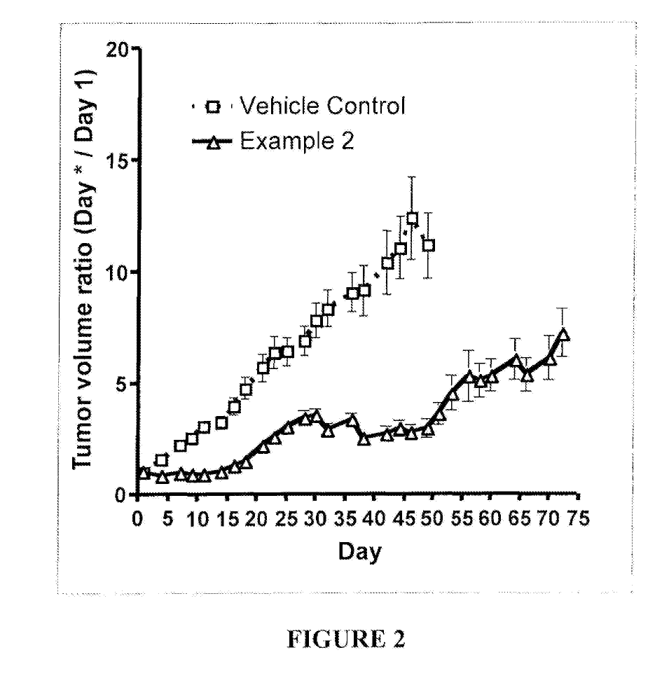

Tumour growth as well as animal health were monitored for up to 72 days post-day 1 of treatment. The results seen in this experiment (see FIG. 2) clearly show tumour growth inhibition in animals treated with two cycles of Example 2. Significant differences in tumour growth between Example 2 treated (n=64) and vehicle treated (n=20) animals were observed as early as day 4 (p<0.001; unpaired t-test; Prism® analysis) through to Day 70.

### (B) (ii) In Vivo Studies for Combinations of (a), (b) and (c)

(a) Example 2 Treatment in Combination with Carboplatin (Combination Partner (b)) and Gemcitabine (Combination Partner (c)).

### Tumour Growth Inhibition:

Balb/c nu/nu mice bearing cisplatin resistant A2780Cis solid orthotopic tumours were treated with compound Example 2 at 24 mg/kg, carboplatin at 50 mg/kg and gemcitabine at 12.5 mg/kg. Animals were i.v. dosed with a single dose of carboplatin, two weekly doses of Example 2 and two weekly doses of gemcitabine. Each cycle of Example 2 and gemcitabine was dosing on days 1 and 8; and each cycle of carboplatin was dosing on day 1.

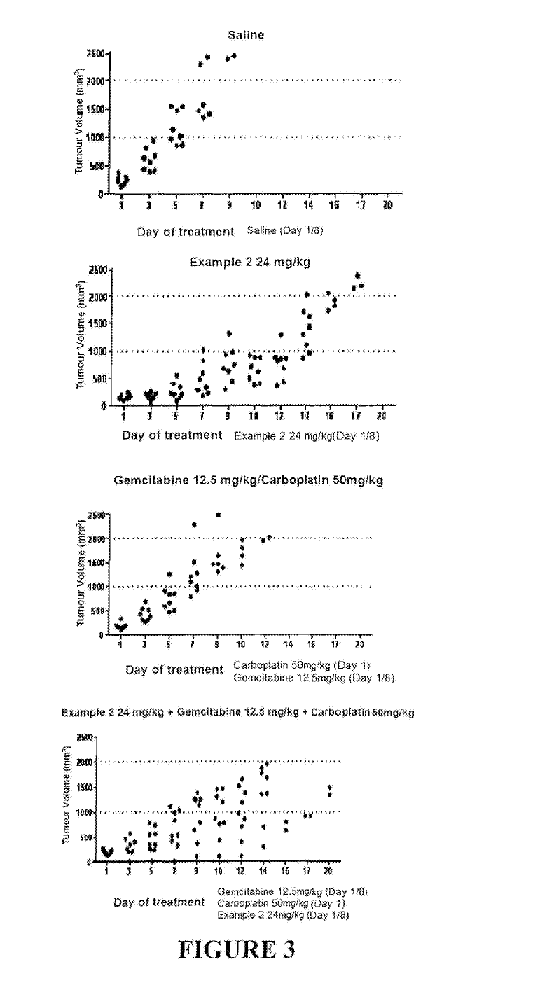

Tumour growth as well as animal health were monitored for up to 21 days post-day 1 of treatment. The results seen in this experiment (see FIG. 3) clearly show tumour growth inhibition in animals treated with the triplet combination compared to carboplatin/gemcitabine doublet combination treatment.

### Clinical Protocols

### Phase I Clinical Trial

### Trial Design

A standard 3+3 Phase I design was used to determine the recommended dose of compound Example 2 with combination partners (b) and (c). In addition, this design also facilitated the determination of secondary endpoints including progression-free survival (PFS) and adverse event rates. Further correlative endpoints that measure the effect of the drug combination on compound Example 2 pharmacokinetics (PK) were also captured in this trial design.

The patient cohort selected for this Phase I clinical trial were female ovarian cancer subjects with a PFS interval of ≧4 months after first or second line platinum-based chemotherapy.

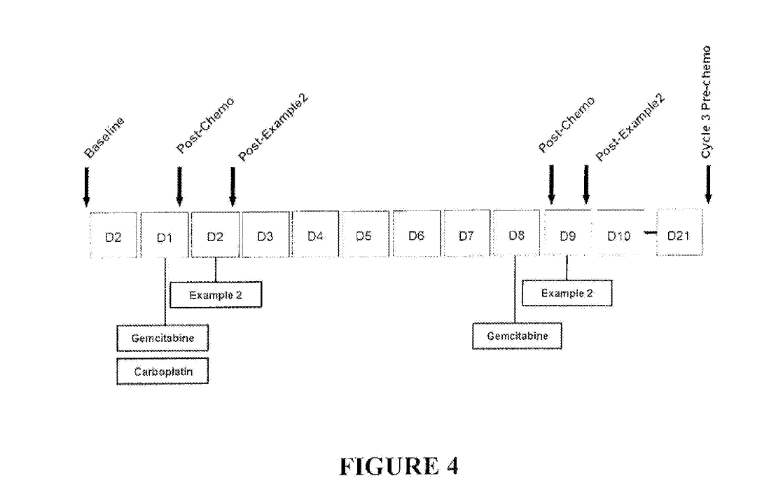

The dosing regime is outlined in FIG. 4. This protocol included compound Example 2 escalation at 12 or 16 mg/m² IV on days 2 and 9, carboplatin AUC4 on day 1, gemcitabine escalation 800 and 1000 mg/m² days 1 and 8. Treatment proceeded across a 21 day cycle for a maximum of 6 cycles. Treatment was followed by single agent maintenance with compound Example 2 at 16 mg/m² for a maximum of 6 additional cycles.

The key outcomes of this trial include the identification of the maximum tolerated dose (MTD) of compound Example 2 and combination partners (b) and (c), dose limiting toxicities and patient response measured according to CA125 levels or RECIST. Additional plasma biomarker studies that measure concentrations of ferritin, interleukin-8, interleukin-16 and macrophage inflammatory protein-1β provide correlative data relating to the PK of compound Example 2.

### Clinical Trial Data

Combination of compound Example 2 with combination partners (b) and (c) is safe and tolerable in ovarian cancer subjects with recurrent ovarian cancer (see Table 2; Table 3 and Table 4).

At dose level 2b no adverse events or dose limiting toxicities were observed. Additionally, a 83% objective response rate was achieved by patients treated at dose level 2b, as measured by CA125 levels of RECIST (see Table 5). As a consequence, dose level 2b was identified as the recommended dose for compound Example 2 and combination partners (b) and (c) for subsequent clinical trials. Specifically, the recommended dose of compound Example 2 and combination partners (b) and (c) is 12 mg/m², AUC4 and 1000 mg/m², respectively (see Table 3).

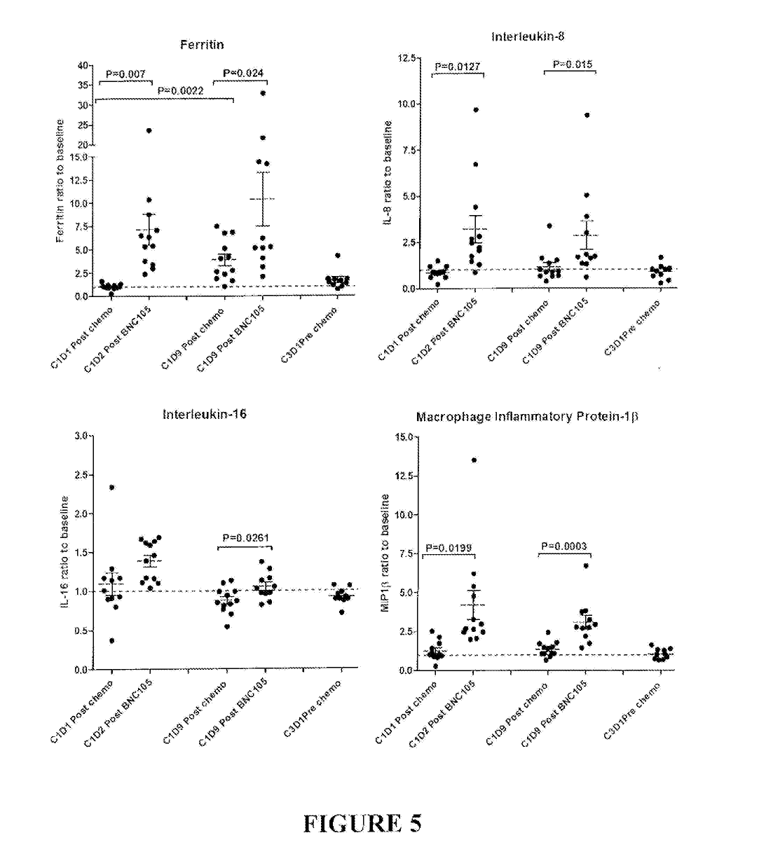

Plasma concentrations of ferritin, interleukin-8, interleukin-16 and macrophage inflammatory protein-1β increase significantly and transiently following the administration of compound Example 2 at 12 mg/m². These data suggest that compound Example 2 at 12 mg/m² reaches a plasma concentration that is sufficient for eliciting a pharmacodynamic response (see FIG. 5).

TABLE 2
Patient baseline characteristics

| | Number of patients |
|---|---|
| ECOG performance status | |
| 0 | 10/15 (67%) |
| 1 | 5/15 (33%) |
| Histologic type | |
| Serous | 12/15 (80%) |
| Other | 3/15 (20%) |
| Number of previous lines of treatment | |
| 1 | 10/15 (67%) |
| 2 | 5/15 (33%) |

TABLE 3
Dose level cohorts and dose limiting toxicities

| | Dose level 1 | Dose level 2a | Dose level 2b |
|---|---|---|---|
| Example 2 | 12 mg/m2 | 16 mg/m2 | 12 mg/m2 |
| Carboplatin | AUC4 | AUC4 | AUC4 |
| Gemcitabine | 800 mg/m2 | 800 mg/m2 | 1000 mg/m2 |
| Number of patients | 6 | 3 | 6 |
| Number of DLTs | 1 | 2 | 0 |

TABLE 4
Grade 3 and 4 toxicities observed across all cycles of treatment

| | Dose level 1 | | Dose level 2a | | Dose level 2b | |
|---|---|---|---|---|---|---|
| | (n = 6) | | (n = 3) | | (n = 60) | |
| | Gr 3 | Gr 4 | Gr 3 | Gr 4 | Gr 3 | Gr 4 |
| Neutropenia | 3 | 2 | 1 | 2 | 5 | |
| Thrombocytopenia | | | | 2 | 2 | |
| Hypertension | 1 | | | | | |
| Hypersensitivity | | | 1 | | | |
| Anaemia | | | | | 1 | |

TABLE 5
Clinical Response

| | Accrued | Objective responses (CA125 or RECIST) | |
|---|---|---|---|
| | N | N | % |
| | 15 | 10 | 68 |
| Dose level | | | |
| 1 | 6 | 4 | 68 |
| 2a | 3 | 1 | 33 |
| 2b | 6 | 5 | 83 |

The claims defining the invention are as follows:

1. A pharmaceutical combination for treating proliferative disease, comprising:
a) a compound of formula (I), or a salt, solvate or prodrug thereof: 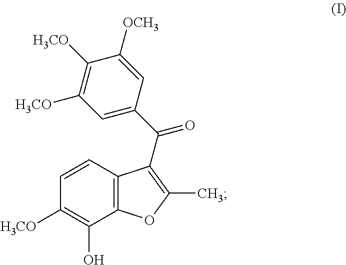 [SMILES: COc1cc(C(=O)c2c(C)oc3c(O)c(OC)ccc23)cc(OC)c1OC]
b) carboplatin or cisplatin; and
c) gemcitabine.

2. A therapeutic protocol to manage ovarian cancer treatment in a female subject, said protocol comprising:
a) selecting an ovarian cancer subject which has had recurrent or persistent ovarian cancer following treatment with platinum-based therapy.
b) administering a combination of effective amounts of (a) a compound of formula (I); (b) carboplatin or cisplatin; and (c) gemcitabine for a defined period of time; and then
c) administering effective amounts of a compound of formula (I) for a defined period of time.

3. The protocol of claim 2 wherein the ovarian cancer subjects are selected following the first relapse after the cessation of primary treatment.

4. The protocol of claim 2 wherein the ovarian cancer subjects are selected following the second relapse after the cessation of secondary treatment.

5. The protocol of claim 2 wherein the ovarian cancer subjects are selected following any additional relapse where it is deemed that the combination therapy may improve patient outcome.

6. The protocol of claim 2 where in the defined period of administration of a compound of formula (I), carboplatin and gemcitabine is from 1 to 6 cycles of drug therapy.

7. The protocol of claim 6 where in the defined period is 21 days with a compound of formula (I) administered at days 2 and 9, carboplatin or cisplatin at day 1 and gemcitabine at days 1 and 8 for up to 6 cycles.

8. The protocol of claim 2 wherein the effective amount of a compound of formula (I) is from 10 to 20 mg/m².

9. The protocol of claim 8 wherein the effective amount of a compound of formula (I) is 12 to 16 mg/m².

10. The protocol of claim 2 wherein the effective amount of carboplatin or cisplatin is AUC4.

11. The protocol of claim 2 wherein the effective amount of gemcitabine is from 500 to 1500 mg/m².

12. The protocol of claim 11 wherein the effective amount of gemcitabine is 800 to 1000 mg/m².

13. A therapeutic kit for the treatment of ovarian cancer in a female subject who has recurrent or persistent ovarian cancer, the kit comprising a compound of formula (I), carboplatin or cisplatin, and gemcitabine, the kit is in a form to dispense a compound of formula (I) in a dosage amount of from 10 to 20 mg/m², carboplatin or cisplatin in a dosage amount of AUC4, and gemcitabine in a dosage amount of from 500 to 1500 mg/m², with instruction for the use of a compound of formula (I), carboplatin or cisplatin and gemcitabine in combination for treating proliferative disease.

14. The use of (a) a compound of formula (I) or a pharmaceutically acceptable salt, solvate or prodrug thereof; (b) carboplatin or cisplatin; and (c) gemcitabine in the treatment of a proliferative disease.

15. The use of (a) a compound of formula (I) or a pharmaceutically acceptable salt, solvate or prodrug thereof; (b) carboplatin or cisplatin; and (c) gemcitabine in the manufacture of a medicament for the treatment of a proliferative disease.

16. A (a) compound of formula (I); (b) carboplatin or cisplatin; and (c) gemcitabine for use in treating an ovarian cancer patient with recurrent or persistent ovarian cancer, wherein a compound of formula (I), carboplatin or cisplatin, and gemcitabine are administered to the subject for a defined period followed by a compound of formula (I) for a defined period.

17. A combination, protocol, kit or use according to any one of claims 1 to 16 wherein a compound of formula (I) is a compound represented by the following: [chemical structure]
